(19) United States
(12) Patent Application Publication
(10) Pub. No.: US 2011/0247119 A1
(43) Pub. Date: Oct. 13, 2011

(54) WELDING MASK

(75) Inventor: Chen-San Cheng, Kaohsiung City (TW)

(73) Assignee: POLISON CORPORATION, Kaohsiung City (TW)

(21) Appl. No.: 12/755,473

(22) Filed: Apr. 7, 2010

(51) Int. Cl. A61F 9/06

(52) U.S. Cl. 2/8.5

(57) ABSTRACT

A welding mask includes a mask body formed with a viewing window, and an eye shield assembly including a window frame, a lens and a resilient positioning frame. The window frame is connected to the mask body, surrounds the viewing window, and includes an inwardly projecting stop and an opposing pair of inwardly projecting retaining portions disposed rearwardly of the stop. The lens is disposed removably between the stop and the retaining portions. The positioning frame is disposed removably between the lens and the retaining portions. The positioning frame defines a convex surface facing a direction away from the lens, and abuts against the retaining portions to thereby result in resilient deformation of the positioning frame and enable the positioning frame to press the lens against the stop.

### BACKGROUND OF THE INVENTION

1. Field of the Invention

The present invention relates to a welding mask, more particularly to a welding mask that can be assembled without damaging a lens thereof.

2. Description of the Related Art

Referring to FIGS. 1 and 2, a conventional welding mask 1 includes a mask body 11 and an eye shield assembly 12. The eye shield assembly 12 includes a window frame 121 connected to the mask body 11, a lens 125 disposed removably in the window frame 121, and a positioning frame 126 for positioning the lens 125 within the window frame 121. The window frame 121 includes a flange 122 projecting inwardly from a front edge of the window frame 121, and a pair of retaining portions 123 disposed at opposing lateral rear edges of the window frame 121. The positioning frame 126 includes an opposing pair of horizontal segments 128, and an opposing pair of lateral pressing segments 127. The positioning frame 126 is disposed removably in the window frame 121 between the retaining portions 123 and the lens 125, and the horizontal segments 128 thereof abut against the lens 125 to press the lens 125 against the flange 122 of the window frame 121.

During removal of the positioning frame 126 and the lens 125 from the window frame 121, a user exerts force upon the pressing segments 127 to result in resilient deformation of the horizontal segments 128 (depicted by the phantom lines in FIG. 2) for releasing abutting engagement between the pressing segments 127 and the retaining portions 123 to permit the removal of the positioning frame 126 and the lens 125 from the window frame 121. However, the lens 125 is subjected to additional force from the horizontal segments 128 due to the deformation of the horizontal segments 128, and thus, the lens 125 is easily scratched or broken. Further, since the pressing segments 127 are fitted within the retaining portions 123, it is inconvenient to press the pressing segments 127.

### SUMMARY OF THE INVENTION

Therefore, an object of the present invention is to provide a welding mask that is relatively easy to assemble and disassemble and that can be assembled without damaging a lens thereof.

According to the present invention, a welding mask comprises a mask body formed with a viewing window, and a first eye shield assembly. The first eye shield assembly includes a first window frame, a first lens and a resilient first positioning frame.

The first window frame is connected to the mask body and surrounds the viewing window. The first window frame includes an inwardly projecting first stop and an opposing pair of inwardly projecting first retaining portions that are spaced apart from each other and that are disposed rearwardly of and spaced apart from the first stop. The first lens is disposed removably in the first window frame between the first stop and the first retaining portions. The first positioning frame is disposed removably in the first window frame between the first lens and the first retaining portions. The first positioning frame defines a convex surface that faces a direction away from the first lens, and includes an opposing pair of first abutting segments and an opposing pair of first connecting segments.

The first abutting segments are spaced apart from each other, and each of the first abutting segments has opposite end portions. The first connecting segments are spaced apart from each other, and each of the first connecting segments interconnects adjacent ones of the opposite end portions of the first abutting segments. Each of the first abutting segments abuts against a respective one of the first retaining portions of the first window frame to thereby result in resilient deformation of the first positioning frame between the first lens and the first retaining portions and enable the first positioning frame to press the first lens against the first stop.

At least one of the first abutting segments has a first press part for exertion of a force to deform resiliently the first abutting segment for releasing abutting engagement between the first abutting segment and the respective one of the first retaining portions to permit removal of the first positioning frame and the first lens from the first window frame.

### BRIEF DESCRIPTION OF THE DRAWINGS

Other features and advantages of the present invention will become apparent in the following detailed description of the preferred embodiments with reference to the accompanying drawings, of which:

FIG. 1 is an exploded perspective view of a conventional welding mask;

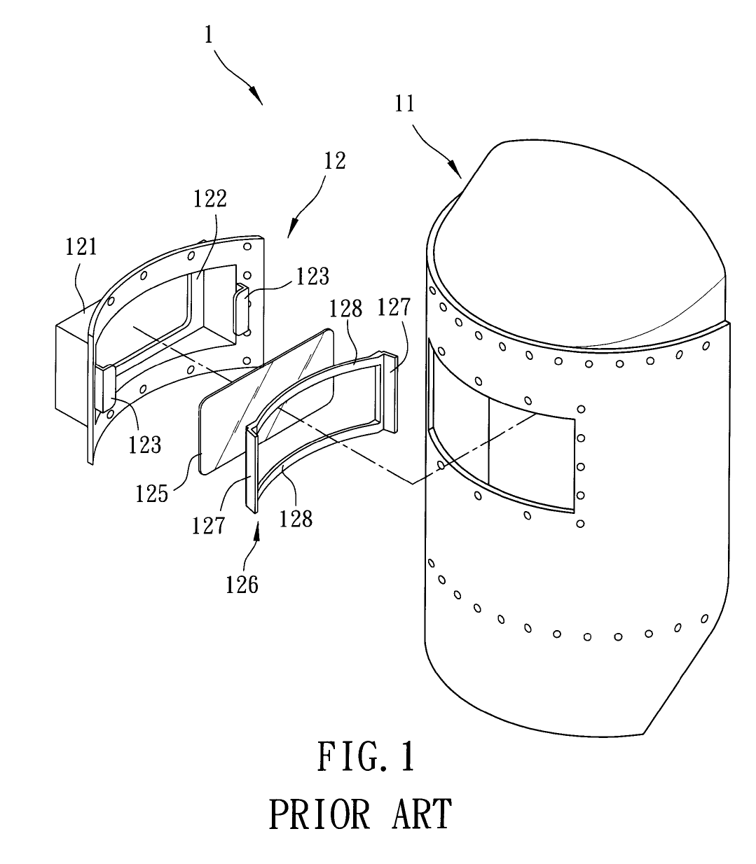

FIG. 2 is a top sectional view of the conventional welding mask;

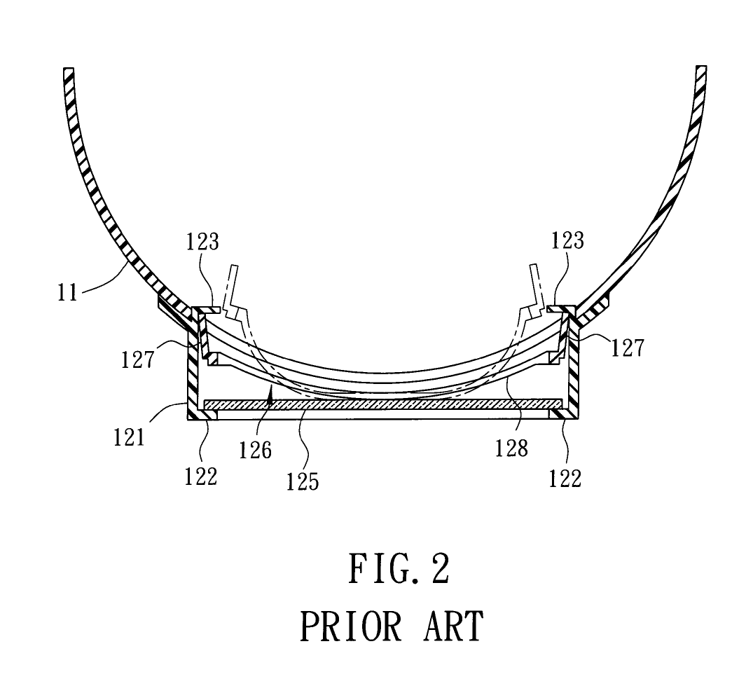

FIG. 3 is an exploded perspective view of the first preferred embodiment of a welding mask according to the present invention;

FIG. 4 is a top sectional view of the welding mask of the first preferred embodiment;

FIG. 5 is an exploded perspective view of the second preferred embodiment of a welding mask according to the present invention; and

FIG. 6 is a perspective view illustrating a second eye shield assembly of the welding mask of the second preferred embodiment at a non-use position.

### DETAILED DESCRIPTION OF THE PREFERRED EMBODIMENTS

Before the present invention is described in greater detail, it should be noted that like elements are denoted by the same reference numerals throughout the disclosure.

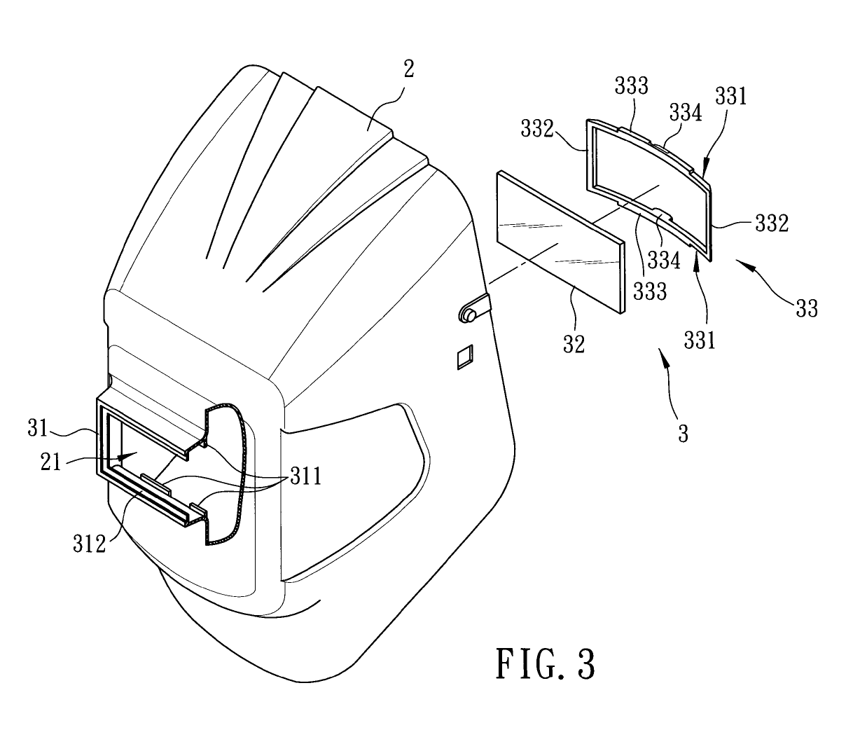

Referring to FIG. 3, the first preferred embodiment of a welding mask of this invention includes a mask body 2 formed with a viewing window 21, and a first eye shield assembly 3. In this embodiment, the viewing window 21 is sized to prevent obstruction of field of vision of a user during welding. In practice, the view window 21 can be also sized according to manufacturing cost or other requirements.

The first eye shield assembly 3 includes a first window frame 31 connected to the mask body 2 and surrounding the viewing window 21, a first lens 32 disposed removably in the first window frame 31, and a resilient first positioning frame 33 abutting against the first lens 32 to position the first lens 32 in the first window frame 31.

The first window frame 31 includes an inwardly projecting first stop 312, and two pairs of inwardly projecting first retaining portions 311 that are disposed rearwardly of and spaced apart from the first stop 312. One pair of the first retaining portions 311 are disposed on a top inner side of the first window frame 31, and the other pair are disposed on a bottom inner side of the first window frame 31. The first retaining portions 311 in the same pair are horizontally spaced apart from each other.

The first positioning frame 33 is disposed removably in the first window frame 31 between the first lens 32 and the first retaining portions 311. The first positioning frame 33 defines a convex surface that faces a direction away from the first lens 32, and includes an opposing pair of first abutting segments 331 and an opposing pair of first connecting segments 332. The first abutting segments 331 are vertically spaced apart from each other, and each of the first abutting segments 331 has opposite end portions. The first connecting segments 332 are horizontally spaced apart from each other, and each of the first connecting segments 332 interconnects adjacent ones of the opposite end portions of the first abutting segments 331.

Each of the first abutting segments 331 includes a first tab part 333 corresponding to one pair of the first retaining portions 311, and a first press part 334 that projects away from the first lens 32 and that is located midway of the respective one of the first abutting segments 331. When the welding mask is assembled, the first tab part 333 abuts against a respective pair of the first retaining portions 311, and the first press part 334 is disposed between the respective pair of the first retaining portions 311.

It should be noted that, in this embodiment, the first lens 32 is made of a material capable of blocking an intense light, and the first window frame 31 can be made as an integral part of the mask body 2. In practice, the first window frame 31 may be manufactured as a separate component that is subsequently connected to the mask body 2 by screwing or gluing. The configuration of the first window frame 31 is not limited to the disclosure of this embodiment. Moreover, the number of the first retaining portions 311 and the first tab parts 333 is only for exemplary purposes, and variations of the number and disposition thereof should be apparent to those who are skilled in the art.

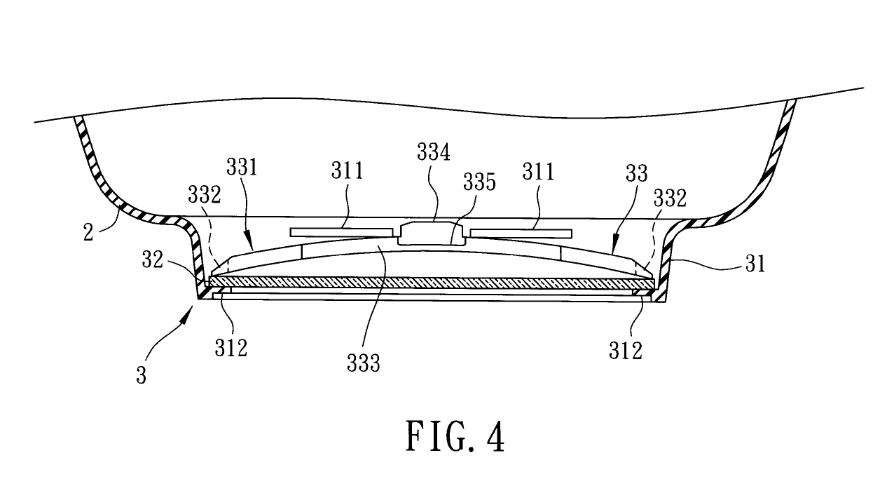

Referring to FIGS. 3 and 4, during assembly of the welding mask, the first lens 32 is placed in the first window frame 31 between the first stop 312 and the first retaining portions 311 first. Then, the first positioning frame 33 is placed in the first window frame 31 in a manner that the convex surface of the first positioning frame 33 faces the direction away from the first lens 32. Each of the first press parts 334 is pressed toward a direction facing the other one of the first press parts 334 to deform resiliently the first abutting segments 331 for placing the first positioning frame 33 between the first lens 32 and the first retaining portions 311 and to thereby result in abutting engagement between the first tab parts 333 and the first retaining portions 311. Therefore, each of the first tab parts 333 abuts against the respective pair of the first retaining portions 311 of the first window frame 31 to thereby result in resilient deformation of the first positioning frame 33 between the first lens 32 and the first retaining portions 311 and enable the first connecting segments 332 of the first positioning frame 33 to press the first lens 32 against the first stop 312.

Preferably, each of the first abutting segments 331 further has an outer side formed with a first recess 335 that is registered with the first press part 334. When pressing the first press part 334, the first recess 335 allows the user to place a finger on the first press part 334 to facilitate exertion of a force to deform resiliently the first abutting segments 331.

During removal of the first lens 32, each of the first press parts 334 is also pressed toward the direction facing the other one of the first press parts 334. Accordingly, the first press parts 334 are subjected to a force to deform resiliently the first abutting segments 331 for releasing abutting engagement between the first tab parts 335 and the first retaining portions 311 to permit removal of the first positioning frame 33 and the first lens 32 from the first window frame 31.

Since the convex surface of the first positioning frame 33 faces the direction away from the first lens 32, the resilient deformation of the first positioning frame 33 has a tendency toward the direction away from the first lens 32. Therefore, the first lens 32 can be prevented from scratching and damaging attributed to the first positioning frame 33 when pressing the first press parts 334. Moreover, since each of the first press parts 334 is located midway of the respective one of the first abutting segments 331 and is not obstructed by other components, the first press parts 334 facilitate the user to exert a force to deform resiliently the first abutting segments 331.

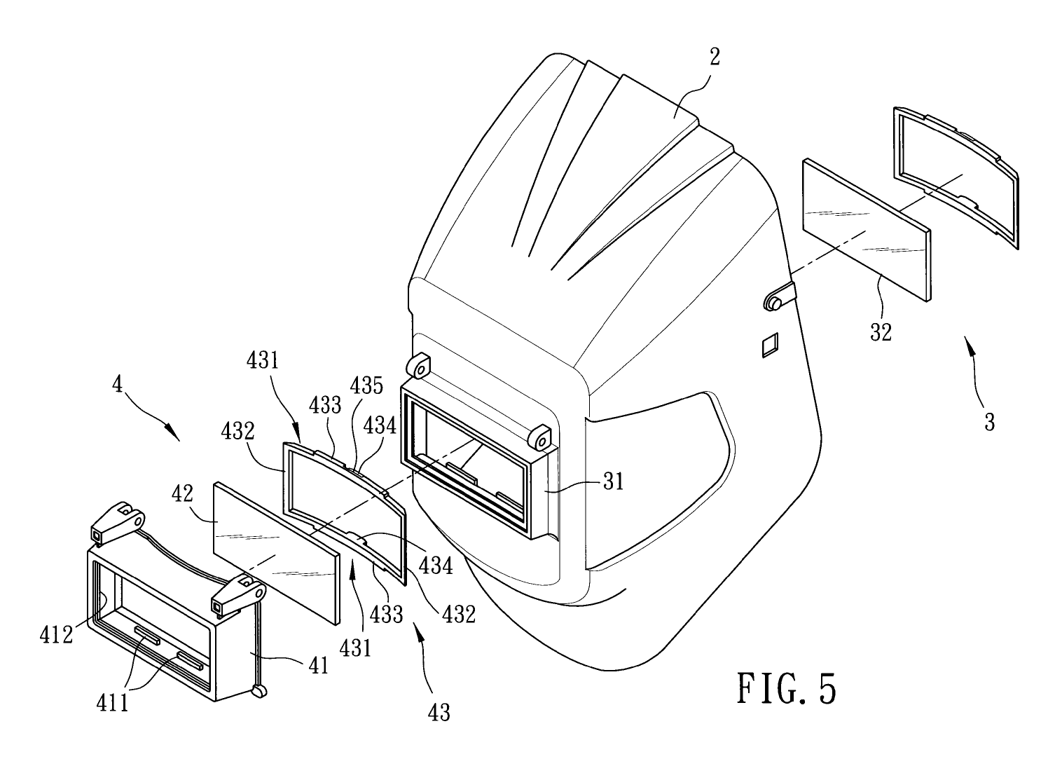

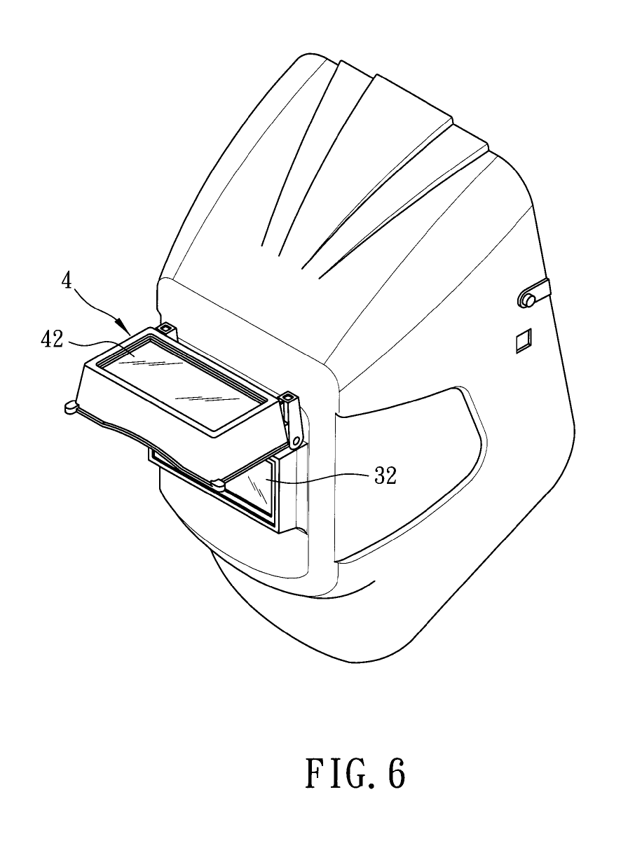

Referring to FIG. 5, the second preferred embodiment of the welding mask of this invention is shown to be similar to the first preferred embodiment. In this embodiment, the welding mask further includes a second eye shield assembly 4 coupled to the mask body 2 of the welding mask. In other embodiments, the second shield assembly 4 may be coupled to the first window frame 31 of the first eye shield assembly 3. The second eye shield assembly 4 includes a second window frame 41 pivoted to the mask body 2, a second lens 42 disposed removably in the second window frame 41, and a second resilient positioning frame 43 abutting against the second lens 42 to position the second lens 42 in the second window frame 41. The second eye shield assembly 4 is movable relative to the first eye shield assembly 3 between a use position, where the second lens 42 is disposed in front of the first lens 32, and a non-use position, where the second lens 42 is moved away from the first lens 32 as shown in FIG. 6.

The second window frame 41 includes an inwardly projecting second stop 412, and two pairs of inwardly projecting second retaining portions 411 (only two of the second retaining portions 411 are visible due to the angle of view) that are disposed rearwardly of and spaced apart from the second stop 412. One pair of the second retaining portions 411 are disposed on a top inner side of the second window frame 41, and the other pair are disposed on a bottom inner side of the second window frame 41. The second retaining portions 411 in the same pair are horizontally spaced apart from each other.

The second positioning frame 43 is disposed removably in the second window frame 41 between the second lens 42 and the second retaining portions 411. The second positioning frame 43 defines a convex surface that faces a direction away from the second lens 42, and includes an opposing pair of second abutting segments 431 and an opposing pair of second connecting segments 432. The second abutting segments 431 are vertically spaced apart from each other, and each of the second abutting segments 431 has opposite end portions. The second connecting segments 432 are horizontally spaced apart from each other, and each of the second connecting segments 432 interconnects adjacent ones of the opposite end portions of the second abutting segments 431.

Each of the second abutting segments 431 includes a second tab part 433 corresponding to one pair of the second retaining portions 411, and a second press part 434 that projects away from the second lens 42 and that is located midway of the respective one of the second abutting segments 431. When the second eye shield assembly 4 is assembled, the second tab part 433 abuts against a respective pair of the second retaining portions 411, and the second press part 434 is disposed between the respective pair of the second retaining portions 411. Each of the second abutting segments 431 further has an outer side formed with a second recess 435 that is registered with the second press part 434. When pressing the second press part 434, the second recess 435 allows the user to place a finger on the second press part 434 to facilitate exertion of a force to deform resiliently the second abutting segments 431.

It should be noted that, in this embodiment, the first lens 32 is a transparent lens and the second lens 42 is capable of blocking an intense light. Therefore, the user can move the second eye shield assembly 4 to the use position for blocking an intense light during a welding operation. Aside from the welding operation, the user can implement other operations by moving the second eye shield assembly 4 to the non-use position without taking off the welding mask. Since the assembly procedure of the second eye shield assembly 4 is similar to that of the first preferred embodiment, further details of the assembly procedure will be omitted herein for the sake of brevity.

In summary, the convex surfaces of the first and second positioning frames 33, 43 face the directions away from the first and second lenses 32, 42, respectively. Therefore, when assembling or removing the first and second lenses 32, 42, the first and second positioning frames 33, 43 will not apply additional force on the first and second lenses 32, 42 to thereby prevent the first and second lenses 32, 42 from scratching and even breaking. Moreover, the configuration of the first and second press parts 334, 434 facilitates the assembly and removal of the first and second positioning frames 33, 43.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A welding mask, comprising:
a mask body formed with a viewing window; and
a first eye shield assembly including
a first window frame connected to said mask body and surrounding said viewing window, said first window frame including an inwardly projecting first stop and an opposing pair of inwardly projecting first retaining portions that are spaced apart from each other and that are disposed rearwardly of and spaced apart from said first stop,
a first lens disposed removably in said first window frame between said first stop and said first retaining portions, and
a resilient first positioning frame disposed removably in said first window frame between said first lens and said first retaining portions, said first positioning frame defining a convex surface that faces a direction away from said first lens, and including
an opposing pair of first abutting segments that are spaced apart from each other, each of said first abutting segments having opposite end portions, and
an opposing pair of first connecting segments that are spaced apart from each other, each of said first connecting segments interconnecting adjacent ones of said opposite end portions of said first abutting segments,
each of said first abutting segments abutting against a respective one of said first retaining portions of said first window frame to thereby result in resilient deformation of said first positioning frame between said first lens and said first retaining portions and enable said first positioning frame to press said first lens against said first stop,
at least one of said first abutting segments having a first press part for exertion of a force to deform resiliently said first abutting segment for releasing abutting engagement between said first abutting segment and the respective one of said first retaining portions to permit removal of said first positioning frame and said first lens from said first window frame.

2. The welding mask as claimed in claim 1, wherein each of said first abutting segments has a first tab part for abutting against the respective one of said first retaining portions, and said first press part projecting away from said first lens.

3. The welding mask as claimed in claim 2, wherein said first press part is located midway of the respective one of said first abutting segments.

4. The welding mask as claimed in claim 3, wherein each of said first abutting segments further has an outer side formed with a first recess that is registered with said first press part.

5. The welding mask as claimed in claim 1, further comprising a second eye shield assembly that is coupled to one of said mask body and said first window frame and that includes a second lens, said second eye shield assembly being movable relative to said first eye shield assembly between a use position, where said second lens is disposed in front of said first lens, and a non-use position, where said second lens is moved away from said first lens.

6. The welding mask as claimed in claim 5, wherein said second eye shield assembly further includes a second window frame pivoted to said one of said mask body and said first window frame, said second window frame being formed with an inwardly projecting second stop and an opposing pair of inwardly projecting second retaining portions that are spaced apart from each other and that are disposed rearwardly of and spaced apart from said second stop,
said second lens being disposed removably in said second window frame between said second stop and said second retaining portions,
said second eye shield assembly further including a second positioning frame disposed removably in said second window frame between said second lens and said second retaining portions for pressing said second lens against said second stop.

7. The welding mask as claimed in claim 6, wherein said second positioning frame is resilient and defines a convex surface that faces a direction away from said second lens, said second positioning frame including:
an opposing pair of second abutting segments that are spaced apart from each other, each of said second abutting segments having opposite end portions; and
an opposing pair of second connecting segments that are spaced apart from each other, each of said second connecting segments interconnecting adjacent ones of said opposite end portions of said second abutting segments;
each of said second abutting segments having
a second tab part for abutting against a respective one of said second retaining portions of said second window frame to thereby result in resilient deformation of said second positioning frame between said second lens and said second retaining portions and enable said second positioning frame to press said second lens against said second stop,
a second press part for exertion of a force to deform resiliently said second abutting segment for releasing abutting engagement between said second abutting segment and the respective one of said second retaining portions to permit removal of said second positioning frame and said second lens from said second window frame, said second press part projecting away from said second lens, and
an outer side formed with a second recess that is registered with said second press part.

8. The welding mask as claimed in claim 7, wherein said second press part is located midway of the respective one of said second abutting segments.

9. A welding mask, comprising:
a mask body formed with a viewing window;
a first eye shield assembly including a first window frame connected to said mask body and surrounding said viewing window, and a first lens disposed in said first window frame; and
a second eye shield assembly coupled to one of said mask body and said first window frame, said second eye shield assembly being movable relative to said first eye shield assembly between a use position and a non-use position, and including
a second window frame coupled to said one of said mask body and said first window frame, said second window frame being formed with an inwardly projecting stop and an opposing pair of inwardly projecting retaining portions that are spaced apart from each other and that are disposed rearwardly of and spaced apart from said stop,
a second lens disposed removably in said second window frame between said stop and said retaining portions, said second lens being disposed in front of said first lens when said second eye shield assembly is at the use position, and being moved away from said first lens when said second eye shield assembly is at the non-use position, and
a resilient positioning frame disposed removably in said second window frame between said second lens and said retaining portions, said positioning frame defining a convex surface that faces a direction away from said second lens, and including
an opposing pair of abutting segments that are spaced apart from each other, each of said abutting segments having opposite end portions, and
an opposing pair of connecting segments that are spaced apart from each other, each of said connecting segments interconnecting adjacent ones of said opposite end portions of said abutting segments,
each of said abutting segments abutting against a respective one of said retaining portions of said second window frame to thereby result in resilient deformation of said positioning frame between said second lens and said retaining portions and enable said positioning frame to press said second lens against said stop,
at least one of said abutting segments having a press part for exertion of a force to deform resiliently said abutting segment for releasing abutting engagement between said abutting segment and the respective one of said retaining portions to permit removal of said positioning frame and said second lens from said second window frame.

10. The welding mask as claimed in claim 9, wherein each of said abutting segments has a tab part for abutting against the respective one of said retaining portions, and said press part projecting away from said second lens.

11. The welding mask as claimed in claim 10, wherein said press part is located midway of the respective one of said abutting segments.

12. The welding mask as claimed in claim 11, wherein each of said abutting segments further has an outer side formed with a recess that is registered with said press part.
